(19) United States
(12) Patent Application Publication
(10) Pub. No.: US 2024/0177300 A1
(43) Pub. Date: May 30, 2024

(54) METHOD, APPARATUS AND SYSTEM FOR IMAGE-TO-IMAGE TRANSLATION

(71) Applicant: L'oréal, Paris (FR)

(72) Inventors: Ruowei JIANG, Toronto (CA); Brendan DUKE, Toronto (CA); Parham AARABI, Richmond Hill (CA)

(73) Assignee: L'oréal, Paris (FR)

(21) Appl. No.: 17/994,792

(22) Filed: Nov. 28, 2022

(51) Int. Cl. G06T 7/00; G16H 30/40

(52) U.S. Cl. CPC G06T 7/0012; G16H 30/40; G06T 2207/20081; G06T 2207/20084; G06T 2207/30088

(57) ABSTRACT

A method, apparatus and system according to embodiments provide image-to-image translations such as synthesis of ultraviolet (UV) images from input images in a RGB (red, green blue) color model. In an embodiment, a trained generator generates overlapping UV patch images from overlapping RBG patch images extracted from an input image. The overlapping UV patch images are blended using a Gaussian weighting factor applied to overlapping pixels having a same location in the input image. The Gaussian blending distributes weights to pixels relative to the pixel's distance to the center of its patch, with weighting being highest at the center.

### FIELD

This disclosure relates to image processing using neural networks and more particularly to image-to-image translation, synthesizing output images such as ultraviolet (UV) images from RGB (red, green, blue) color model images. UV images are useful as images in their own right and can be used for, inter alia, analysis of skin such as for one or more skin conditions or injury, or skin synthesis such as for simulation of aging.

### BACKGROUND

Over the last decade, machine learning techniques have shown substantial improvements in real applications. Skin assessment, including skin disease identification and clinical sign scoring has been one of the major applied domains that witnessed such success. Additionally, skin synthesis (such as aging), has been first explored in physical-based and prototype-based approaches. More recently, such works are often achieved through synthesized face images modeled by generative models for image-to-image translation that have shown success in simulating changes on wrinkles and other aging characteristics.

Commonly available cameras such as found in many electronic systems such as smartphones, tablets, laptops, webcams, or other consumer devices produce images, such as selfie photos, using a RGB color model. The RGB color model is an additive color model based upon the three primary colors red, green, blue from the visible portion of the light spectrum. Ultraviolet photography records images by using radiation from only the UV portion of the light spectrum. As UV radiation is invisible to the eye, UV images have no color and comprise grayscale images. UV images can serve a number of purposes, particularly scientific and medical purposes. While useful in their own right as a translation from color photography, diagnostic medical images, as an example, are useful for detecting skin conditions or evidence of injury.

It is desired to have a method and system for image-to-image translation such as skin image simulation to synthesize ultraviolet (UV) images from original RGB images.

### SUMMARY

There is provided a method and system to synthesize images from one domain to another, such as from an RGB image domain to a UV image domain. In an embodiment, a generator translates overlapping input image patches to overlapping output image patches in an output domain. Gaussian-based patch blending is used to construct the output image from the overlapping output image patches. The patch blending weighs the overlapping pixels at the same coordinates (e.g. relative to the input image or the output image) among multiple generated patches based on each pixel's distance to its patch center point. In an embodiment, the generator is trained using cycle consistent training of a pair of generators translating in opposite directions. While embodiments are described to translate images between the UV and RGB domains, the generators can be trained for other image-to-image translation. In an embodiment, generated images can be provided for additional image processing such as to add an effect thereto, etc.

In an embodiment, the approach takes 0.72 s for a whole face image at resolution 960×720 at inference time and generates realistic-looking ultraviolet images possessing high correspondence with true ultraviolet images.

### BRIEF DESCRIPTION OF THE DRAWINGS

FIG. 1A is an illustration of a training pipeline while FIG. 1B is an illustration of an inference time pipeline, where the trained generator of FIG. 1A is used.

FIG. 2 is an illustration of a Gaussian-based patch blending technique, in accordance with an embodiment.

FIGS. 3 and 4 are arrays of images showing qualitative comparison results.

FIG. 5 is a graph showing quantitative comparison results.

FIG. 6 is an illustration of a computer network providing an environment for various aspects according to embodiments herein.

FIG. 7 is a block diagram of a computing device of the computer network of FIG. 6.

FIG. 8 is a flowchart of operations in accordance with an embodiment.

### DETAILED DESCRIPTION

In accordance with an embodiment, there is provided a novel application of skin image simulation to synthesize ultraviolet images from original RGB images. Ultraviolet (UV) images, which are often obtained using radiation from the UV spectrum only, can demonstrate a direct look at the sun damage to the skin. Directly paired translation can be difficult due to the lack of perfectly aligned inputs and outputs. Therefore, in embodiment, connections are established in a neural network via a self-supervised learning cycle such. In an embodiment, there are two generators of a generative adversarial network (GAN) model trained via cycle consistency to translate from the RGB and UV domains in both directions on RGB and UV skin images. In an embodiment, the training image sets are unpaired images. Training using paired images can be employed.

To ensure high-resolution results, in an embodiment, the generators are trained with skin patches (such as extracted from a face image) and the patches are blended at the inference time (for example, to reconstruct the face image in UV or RGB). But GAN generated images can often be inconsistent with pixel shifts on the original images, which causes aliasing in the generator. With slight differences on the same target pixel in different patches, gridding and color inconsistency issues can frequently appear when using trivial blending methods.

Linear blending approaches to address such problems can work well on simple patch blending but often fail on densely scanned original images—e.g. where a same pixel from an original image appears in multiple overlapping patches. It is observed that due to the central-focused network design, GANs can often have higher quality pixels in the central region of each image it generates (e.g., a patch of another image) compared to the corners. In an embodiment, introduced is a novel blending technique that merges the same pixel from multiple patches by weighing the pixels relatively to its distance to the center. Consider a first pixel in a first patch that overlaps with a second pixel in a second patch and where the first pixel is closer to the center of the first patch and the second pixel is closer to the edge (is in a surrounding area) of the second patch. Intuitively, while blending, the first pixel closer to the center would be weighted more than the second pixel in the surrounding area. To make the blending smooth, a Gaussian map is generated for each patch and the Gaussian value is normalized along the same coordinate for overlapped patches. A blended pixel is the weighted average of all pixels in all overlapped patches at the same coordinates. Effectiveness of the blending technique is demonstrated both qualitatively and quantitatively.

### A. UV-GAN Model

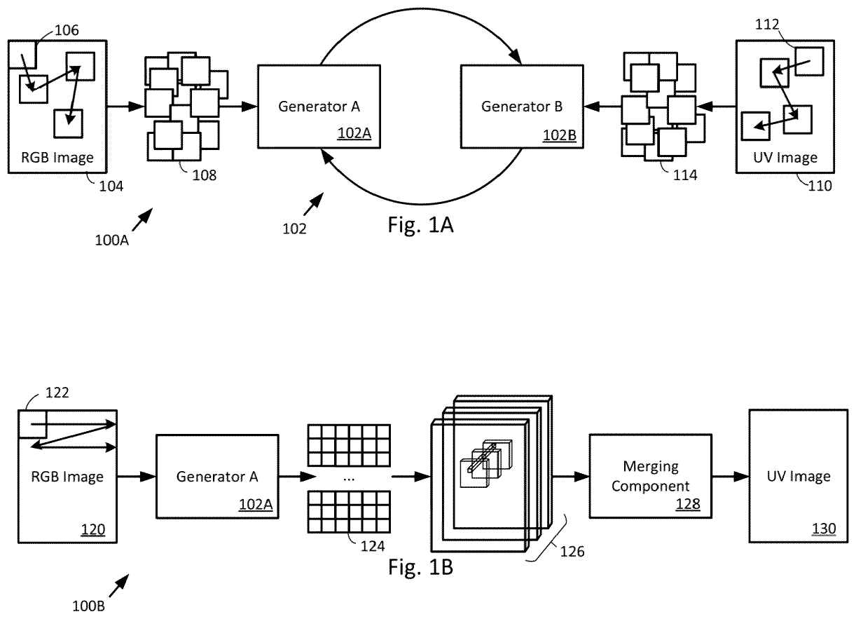

FIG. 1A illustrates an overview of a training pipeline 100A and FIG. 1B illustrates an overview of an inference pipeline 100B having a trained generator from training pipeline 100A, each in accordance with an embodiment. Inference pipeline 100B is for use after the generators of FIG. 1A are trained, such as for testing use or use such as in an application or other manner to obtain translated images.

Regarding FIG. 1A, there is shown a GAN model 102 comprising generator A 102A and generator B 102B for cycle consistent training. To obtain a high-resolution result, a training dataset was prepared as cropped patches of both the original RGB images and the original UV images. In an embodiment, patches of a consistent patch size are cropped at random locations of the original images. Thus, FIG. 1A shows a representative RGB image 104 and a representation of a window 106 used to randomly extract RGB patches 108. RGB patches 108 are extracted (e.g. defined) in response to the window as it is advanced to random locations about RGB image 104 to select groups of pixels within the dimensions of the window. An automated process can be defined to extract the patches for application to train the GAN model 102, for example, to generator A 102A. Similarly a representative UV image 110 is used via a window 112 to define UV patches 114. UV patches 114 are applied to GAN model 102, for example, to generator B 1028. In an embodiment each original image is used to extract N patches (e.g. N=20) but the same count of patches need not be extracted from each image. Patch size and count may vary, for example, with the original resolution of the input images. Each of Generator A and Generator B are useful to for image-to-image translation tasks. In an example, an RGB input image can be translated using Generator A to an UV image. An effect can be applied (e.g. using image processing techniques, but not shown). The image with effect can be translated by Generator B back to an RGB image. Generically, Generator A translates images from a first domain to a second domain and Generator B translates images from the second domain to the first domain.

Patches overlap one another based upon the original location of the patch pixels in the original image. For example, a particular pixel in a first patch overlaps with a particular pixel in a second patch if each of these particular pixels are cropped from the same pixel location in the original image. Overlapping pixels from overlapping output image patches are useful to construct a final output image by blending the overlapping pixel values to construct a single pixel in the output image. In an embodiment, the output image has a same resolution as the input image.

Thus, in an embodiment, at training time, the network 102 is trained to learn the two directions of the image translation via cycle-consistent losses such as using the techniques described in Zhu. J. Y., Park, T., Isola, P., Efros, A. A.: Unpaired image-to-image translation using cycle-consistent adversarial networks (Proceedings of the IEEE international conference on computer vision. pp. 2223-2232 (2017)), incorporated by reference herein in its entirety.

The embodiments of FIGS. 1A and 18 relate to image-to-image translation between RGB and UV domains. Other domain pairs for image-to-image translation can be used such as between a different color model and UV, between female and male gender domains, or between different age domains, etc. While a cycle consistent GAN (ccGAN) training paradigm is shown, another GAN model architecture can be used.

With reference to FIG. 1B, inference pipeline 100B shows a representative RGB image 120 and a (sliding) window 122 with which to extract pixels to define overlapping input patches (not shown). For a whole face image, the sliding window is used to densely scan through the face to generate patches with overlapping coordinates. In this way, the pixels of the input image 120 are sampled to multiple overlapping patches such that the location of a pixel from the input image 120 is at different locations in the overlapping patches. For a particular pixel that has a same location in the original image, its location in an overlapping patch will vary in distance to the center. That is, in one overlapping patch the particular pixel may be located close to the center of the one overlapping patch and in another overlapping patch, the particular pixel may be located further away from the center, for example, closer to an edge of the other overlapping patch.

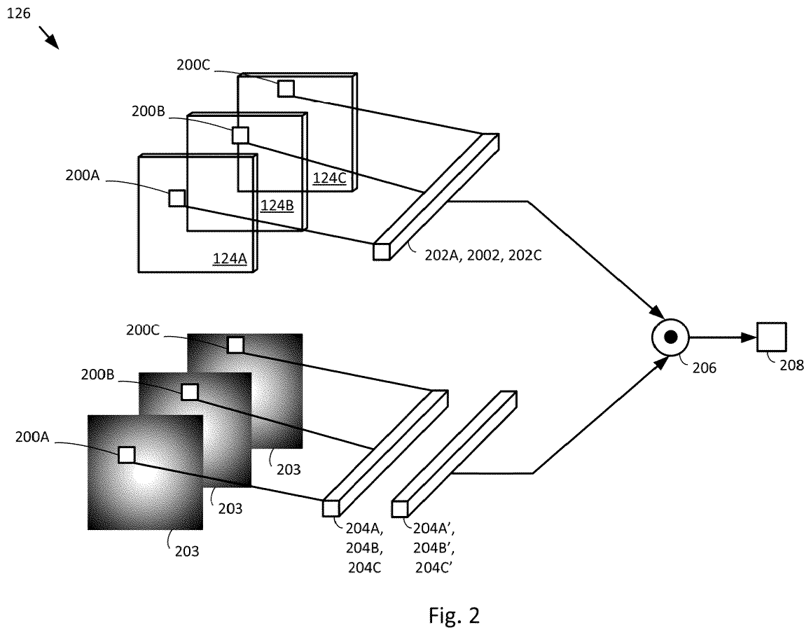

In an embodiment, all the overlapping input patches from RGB image 120 are processed by the trained generator A (i.e. 102A, as trained) to produce overlapping output patches 124. These UV patches overlap in a same manner as the overlapping input images. The pipeline 1008 shows a blending step component 126 that accumulates the (overlapping) pixels corresponding to the same pixel by a separately calculated weighting vector described in FIG. 2. A merging component 128 assembles the blended pixels responsive to the overlapping patches into an output image 130, an UV image that corresponds to the input image 120 translated from the RGB color model to UV. In accordance with an embodiment, through patch based approaches, generator A is configurable for operation (e.g. execution) such as by commonly available consumer devices including smartphones and tablets. Operation can be via a native operating system application or a web-based application.

### B. Patch Blending

One of the disadvantages of patch-based models is the gridding issue (on reconstruction). On the image-to-image translation tasks, the generator tends to focus on the central area of an output image and can be less invariant on the surrounding pixels. One prior solution discloses to densely scan across the complete whole image and take the average of all pixel values (e.g. grayscale pixel values in the illustrated embodiment for an image translated to the UV domain) predicted at the same pixel coordinates. Although this method considerably reduces the gridding effect to a minimum amount with decreasing size of the sliding window, it can still appear when looking closely at the images. Furthermore, the inference time also grows exponentially with more frequent scanning—e.g. to produce small patch sizes with small strides.

To overcome a gridding effect without incurring a computation penalty, in accordance with an embodiment, there is proposed a novel blending method that calculates a weighted vector from overlapping Gaussian masks. The idea is to apply a smoothing vector that weighs the importance of the overlapping pixels based on their respective distances to the center in the generated patches.

FIG. 2 is an illustration of blending component 126 and merging component 128 for operations on representative pixels in overlapping patches 124A, 1248 and 124C. In an embodiment, the blending components provides overlapping patch merging by normalized Gaussian weighting. As shown in FIG. 1B, generator A 102A generates multiple overlapping patches 124. For purpose of illustrating an example, it is assumed that a particular pixel location in RGB image 120 is represented in three overlapping RGB patches that, after the patches are applied to generator A, result in three overlapping UV patches 124A, 124B and 124C. It is understood that more or fewer overlapping patches may result depending on the original location of the particular pixel in RGB image 120, the original image size and the patch generating parameters used for generating the RGB patches.

In the example of FIG. 2, the original pixel location of a pixel in image 120 corresponds to overlapping locations 200A, 2008 and 200C in the three UV patches 124A, 1248 and 124C. According to the present embodiment, the original pixel location of a pixel in image 120 corresponds to the same location in image 130. The pixel values 202A, 2028 and 202C at the overlapping locations 200A. 200B and 2000 are obtained for the blending operation. Three Gaussian masks 203 corresponding to the UV patches 124A, 1248 and 124C are also shown in FIG. 2. For corresponding locations 200A, 2006 and 200C, the UV mask(s) provide blending mask values 204A, 2048 and 204C that are normalized 204A′, 204B′ and 204C′ for use. Normalization is responsive to the scale of the UV domain, for example, the vector is normalized to add up to one. It is understood that separate instances of the mask 203 are shown for illustration purposes but multiple masks are not necessary when computationally performing the operations. Operation 206, a dot product, illustrates an application of the normalized mask values 204A′, 2048′ and 204C′ to the pixel values 202A, 202B and 202C giving a blended pixel value 208 with which to produce UV image 130. Similar operations repeat for all the pixels in the respective patches. Some pixels may be blended with more or fewer other pixels responsive to the presence or not of one or more overlapping patches for the location of the pixel in the original image. In an embodiment, for example so that pixels nearer a corner of the input image are sampled to patches that overlap, an input image may be padded with 0 values for pixels (e.g. a black border) about the border of the image responsive to the size of the sliding window and the stride. The black border pixels don't impact the processing and the border can be discarded when constructing the output image to obtain an image of equal resolution to the original input image.

Hence multiple overlapping patches are merged at inference time to render the output UV image. The normalized vector of Gaussian mask values weights the generated pixel from each patch to produce the merged pixel for the location of the pixel in the output image.

In accordance with an embodiment, there is provided a set of operations showing an overview of the calculation of each pixel. For each pixel, operations normalize along generated Gaussian masks concatenated based on the coordinates (overlapping pixel locations) to achieve one merged pixel. In The following listed operations, there is shown steps to obtain a merged image. The listing denotes pₙ^{i,j} ∈ [symbol] ^{K×K×K×1} as the grayscale nth generated RGB patch processed at image coordinates [i:i+K, j:j+K]. A Gaussian mask g is generated with mean at the center of the patch and variance σ, where a is a hyerarameter of the operations. In an example, variance σ is 1.0.

### Operations to Blend all Patches

1. Initialize g of size K×K with σ(e.g. prepare a Gaussian Mask)
2: Initialize lₐₗₗ∈R^{W×H×N}, Gₐₗₗ∈ [symbol] ^{W×H×N}, I_{merged}∈ [symbol] ^{W×H×1} with all zeros.
3: Scan the input images with a slide window s to get a total of N patches in RGB, with sizeK×K (e.g., extract the RGB patches)
4: For each RGB patch Pₙ^{i,j}∈ [symbol] ^{K×K×1} in N patches:
5: I′ₐₗₗ[i:i+K;j:j+K,n]=Generator(pₙ^{i,j})
6: Gₐₗₗ[i:i+K;j:j+K, n]=g
7: I_{merged}=dot_product(lₐₗₗ, Gₐₗₗ)
8: return I_{merged}

In an embodiment, an image dataset of 491 American women was prepared. The dataset includes 131 Non-Hispanic Euro-American, 138 African American, 116 Hispanic Euro-American and 106 East Asian, covering a wide range of subject age distribution from 18 years old to 80 years old. The dataset contains 6 images for each subject, including one frontal view and two profile views of both RGB images and the UV images. Both the RGB and the UV images are collected using a specific camera device. To obtain specific patches for training and testing the model, patches were prepared from the dataset by randomly cropping 20 patches of resolution 128×128 from each image in the dataset. The dataset was randomly split by 80% and 20% by identity for training and testing set respectively. In the embodiment the network comprises a CycleGAN training setup using a ResNet 9 Block backbone, with ngf 64. After training, an inference pipeline, e.g. in accordance with FIG. 18, using the trained generator according to the embodiment was used to generate images for qualitative and quantitative assessment.

For comparison purposes, additional inference pipelines were prepared using the same trained generator but where patches of different sizes were extracted and used and where the blending and merge components were replaced in accordance with a linear blending approach as disclosed in Cheng, Y. C., Lin, C. H., Lee, H. Y., Ren, J., Tulyakov, S., Yang, M. H.: In&out: Diverse image outpainting via GAN inversion. (Proceedings of the IEEE/CVF Conference on Computer Vision and Pattern Recognition. pp. 11431-11440 (2022), which is incorporated by reference herein in its entirety.) Qualitative results of the model of the embodiment were compared with the linear blending approach used on different slide sizes.

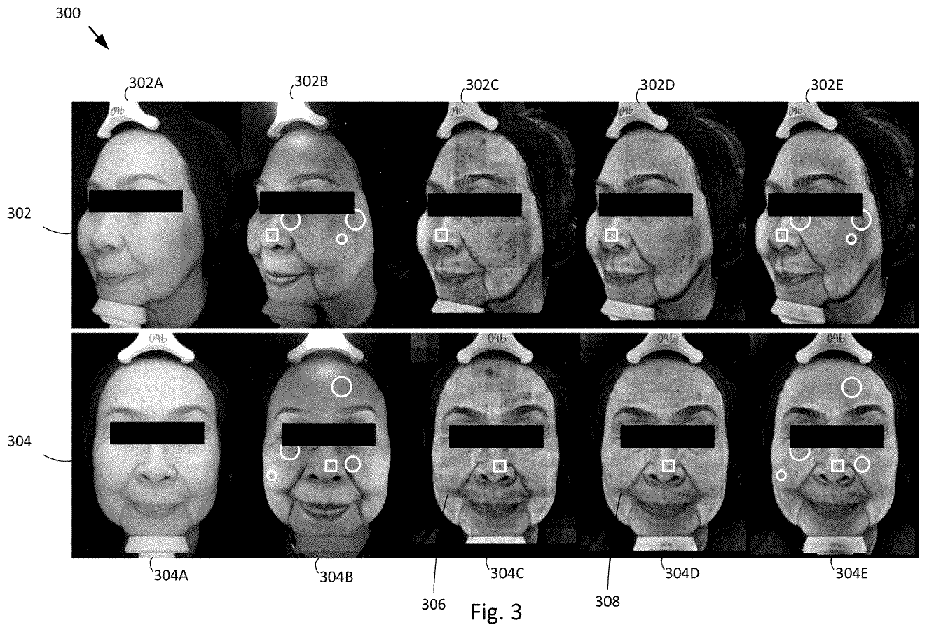

FIG. 3 is an array of images 300 having row 302 of respective images 302A. 302B, 302C, 302D and 302E and row 304 of respective images 304A, 304B, 304C, 304D and 304E. The images are of a same subject from the dataset described herein showing a profile view in row 302 and a frontal or portrait view in row 304. For privacy reasons an eye region mask is added for the present application which mask was not present during use of the images. Images 302A and 304A are original RGB images from the dataset. The RGB images are not shown in color in the present application. Images 302B and 304B are original (ground truth) UV images from the dataset. The sample is a randomly sampled example from the test set. Images 302C and 304C are resulting images from the linear blending using slide ½ patch. Images 302D and 302E are resulting images from the linear blending using slide ¼ patch. Images 302E and 304E are outputs of the model in accordance with the embodiment using a Gaussian blending as described herein.

FIG. 3 shows a strong correspondence in skin features (highlighted in white squares and circles) between generated UV images 302E and 304E in accordance with the teachings herein and the true UV images 302B and 304B in both frontal and profile view.

With the comparison, it is apparent that the method of an embodiment taught herein also generates smoothly blended results compared to the linear blending methods at different sizes of sliding window (images 302C and 304C, and 302D and 304D). Gridding is evident at 306 and 308, in respective patch sizes.

FIG. 4 is an array of images 400 having row 402 of respective images 402A, 402B, 402C, 402D and 402E and row 404 of respective images 404A, 4046, 404C, 404D and 404E. The images are zoomed in relative to the dataset images. The images are of a same subject in FIG. 4 but zoomed in relative to the images of FIG. 3. The array shows a (second) profile view in row 402 and a same profile view in row 404 as was shown in row 304. Images 402A and 404A are original RGB images from the dataset, zoomed in. The RGB images are not shown in color in the present application. Images 402B and 404B are original (ground truth) UV images from the dataset, zoomed in. The sample is a randomly sampled example from the test set. Images 402C and 404C are resulting images from the linear blending using slide ½ patch. Images 402D and 402E are resulting images from the linear blending using slide ¼ patch. Images 402E and 404E are outputs of the model in accordance with the embodiment using a Gaussian blending as described herein.

As shown in FIGS. 3 and 4, while the linear blending methods produce smoother results with less-noticeable gridding by moving with a smaller slide window, the results prepared by the method according to the embodiment described shows superior blending with faster inference speed.

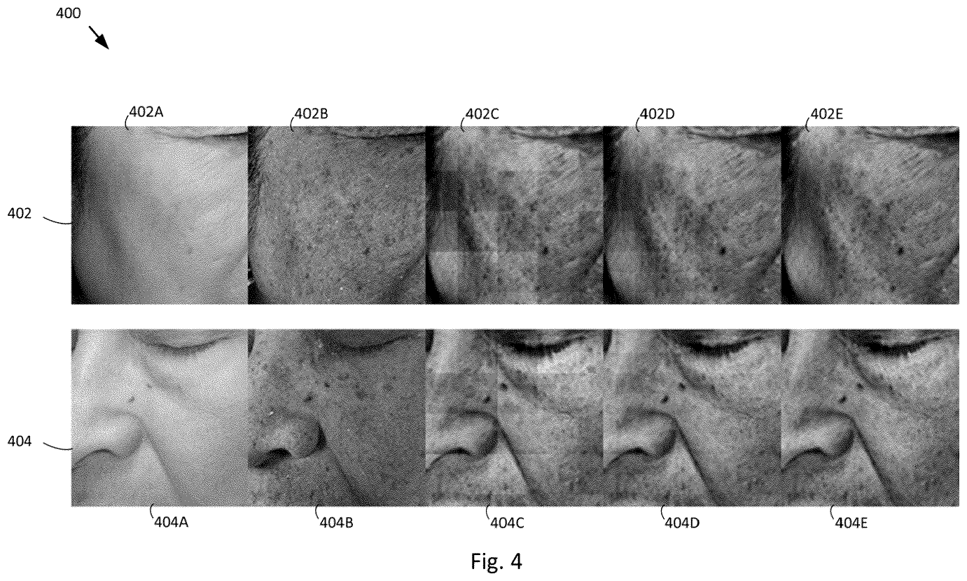

FIG. 5 is a graph showing quantitative comparison results 500. The image quality is evaluated by measuring Frėchet inception distance (FID) between the generated images and the ground truth UV images, which compares the distribution of the images of the two groups. A lower FID indicates a smaller distance between the two distributions. In FIG. 5, the tradeoff between FID and inference time when using the linear blending methods is shown. With slide window size of ½ (502), ¼ 504), and ⅛ patch size (506), FID of 177.24, 121.9, 91.18 and inference time 1.37 s, 4.64 s, 10 s are achieved respectively. The size of the circles in FIG. 5 is proportional. The proposed Gaussian weighted blending (circle 508) achieves the lowest FID of 87.91 with the lowest inference time of 0.72 s. Although with a slide window size of ⅛ patch size (circle 508), a relatively a similar FID is achieved but the inference time is significantly higher.

Thus the comparative examples show typical window slider size=½, ¼, ⅛ patch size and that the number of patches as well as the inference time grow exponentially as the window slider decreases. A reason that dense scanning improves final results with linear blending is that more patches enable the transition between two connecting pixels to be smoother. In an extreme case: sliding with a slide size of 1 pixel was performed, there will be no gridding issue.

With Gaussian blending, as pixels are re-weighted by their importance, such blending will be smooth even with a large window slider size. And therefore such blending enables much faster (e.g. input image) processing.

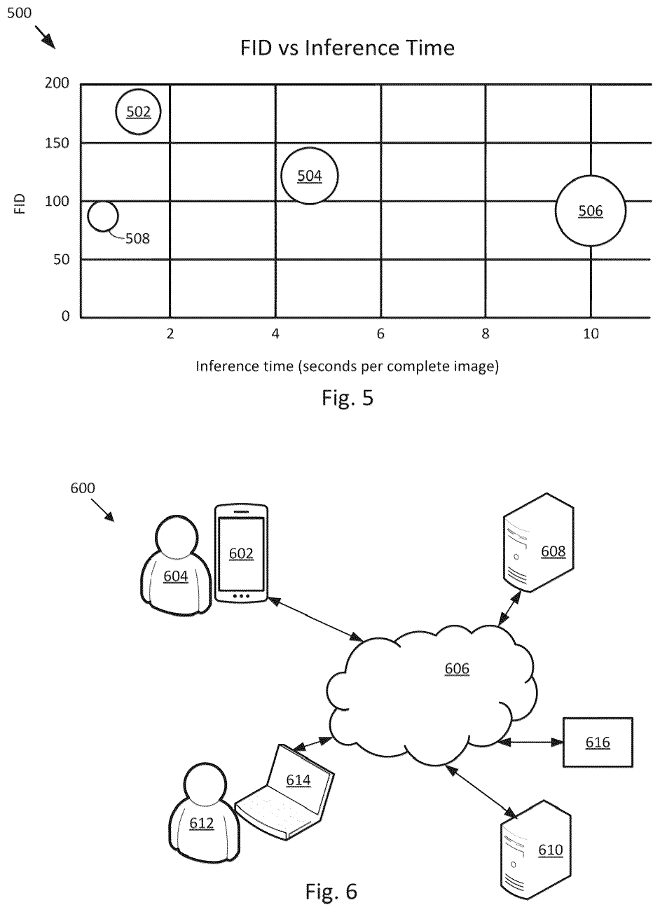

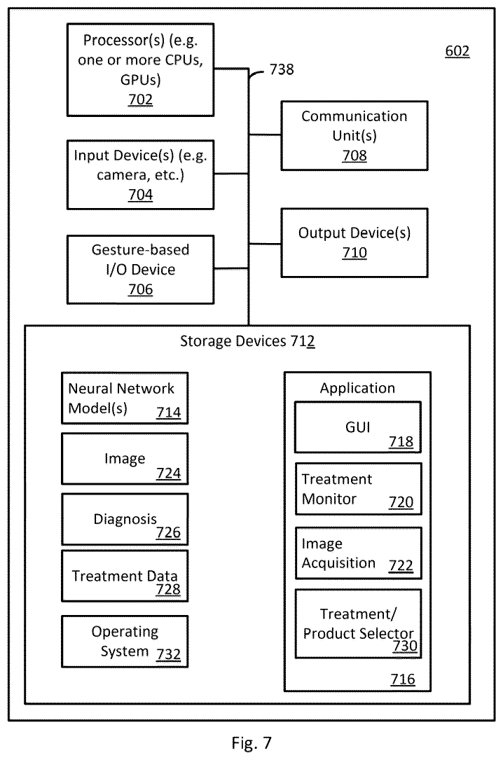

FIG. 6 is an illustration of a computer network providing an environment for various aspects according to embodiments herein. FIG. 7 is a block diagram of a computing device of the computer network of FIG. 6.

FIG. 6 is a block diagram of an example computer network 600 in which a computing device 602 for personal use operated by a user 604 is in communication via a communications network 606 with remotely located server computing devices, namely server 608 and server 610. User 604 may be a consumer and/or a patient of a dermatologist. Also shown is a second user 612 and a second computing device 614 configured for communication via communications network 606. Second user 612 may be a dermatologist, for example. Computing device 602 is for personal use by a user and is not available to the public. Here, the public comprises registered users and/or customers, etc. Computing devices 602 and 614 may also communicate with one another. Device 616 represents a skincare formulation device, for example, a machine to prepare a custom skincare product using a recommended product ingredient. Any of the devices 608 or 610, or 604 or 614 can be enabled to communicate with device 616, directly or indirectly. For example, server 608 or 610 can recommend a product ingredient to a user 612 via device 614 or to user 604 via device 602. The user 612 or 604 can respectively authorize server 610 and/or 608 to communicate the ingredient to device 616. The authorization can be associated with an ecommerce purchase, for example.

Briefly, computing device 602 is configured to perform skin diagnostics. In an embodiment, skin diagnostics is performed on a UV image. A neural network for skin diagnostics may be stored and utilized on board computing device 602 or it may be provided from server 608 such as via a cloud service, web service, etc. from image(s) received from computing device 602. In an embodiment, an inference pipeline for translating and blending UV images from RGB images is provided by at least one of the computing devices of system 600. A neural network to analyze a skin image and produce a scoring result for various skin conditions is described in patent publication US20200170564A1, dated Jun. 4, 2020 of U.S. application Ser. No. 16/702,895, filed Dec. 4, 2019 and entitled “Automatic Image-Based Skin Diagnostics Using Deep Learning”, the entire contents of which is incorporated herein by reference in its entirety.

Thus a skin damage prediction engine can be configured and provided that determines a pixel-wise prediction score for a presence, absence, or severity of one or more skin damage characteristics in the translated UV skin image using one or more convolutional neural network image classifiers. The translated UV image or the RGB input image can be annotated responsive to, for example, the prediction score for a presence, absence, or severity of one or more skin damage characteristics. Other forms of display related to the skin damage characteristics can be provided. A skin damage severity engine can be configured and provided that generates a virtual display including one or more instances of a predicted presence, absence, or severity of the one or more skin damage characteristics responsive to one or more inputs based on the prediction scores for the presence, absence, or severity of at least one skin damage characteristic.

Computing device 602 is configured to communicate with server 610 for example to provide skin diagnostic information and receive product/treatment recommendations responsive to a skin diagnosis. Computing device 602 can be configured to communicate other information regarding the user e.g. age, gender, location, etc. Computing device 602 may be configured to communicate skin diagnostic information (which may include image data) to either or both of server 608 and 610, for example, to store in a data store (not shown). Server 610 (or another server not shown) may provide e-commerce services to sell recommended product(s). Product recommendation can be determine in response to various information including the presence, absence, or severity of at least one skin damage characteristic. Other information may include geographic information such as to determine what products are available based on a location of the user. In an embodiment, a product recommendation comprises a skincare product ingredient. Such an ingredient can be useful to, for example, formulate a customized skincare product. The formulation may comprise an emulsion, ointment, solution or powder, for example. The skincare product ingredient can be transmitted to skincare formulation device 616 that creates the custom skin care product.

Computing device 602 is shown as a handheld mobile device (e.g. a smartphone or tablet). However it may be another computing device such as a laptop, desktop, workstation, etc. Skin diagnosis as described herein may be implemented on other computing device types. Computing device 602 may be configured using one or more native applications or browser-based applications, for example.

Computing device 602 may comprise a user device, for example, to acquire one or more images such as a picture of skin, particularly a face, and process the images to provide skin diagnostics. The skin diagnostics may be performed in association with a skin treatment plan where images are acquired periodically and analyzed to determine skin scores for one or more skin signs. The scores may be stored (locally, remotely or both) and compared between sessions, for ex-ample to show trends, improvement, etc. Skin scores and/or skin images may be accessible to the user 604 of computing device 602 and made available (e.g. via server 608 or communicated (electronically) in another manner via communication network 606) to another user (e.g. second user 612) of computer system 600 such as a dermatologist. Second computing device 614 may also perform skin diagnostics as described. It may receive images from a remote source (e.g. computing device 602, server 608, etc.) and/or may capture images via an optical sensor (e.g. a camera) coupled thereto or in any other manner. A skin diagnostics neural network may be stored and used from second computing device 614 or from server 608 as described.

An application may be provided to perform the skin diagnostics, suggest one or more products and monitor skin changes following one or more application of the product (which may define treatment sessions in a treatment plan) over a time period. The computer application may pro-vide workflow such as a series of instructive graphical user interfaces (GUIs) and/or other user interfaces, which are typically interactive and receive user input, to perform any of the following activities: —skin diagnostics; product recommendation such as for a treatment plan; —product purchase or other acquisition; —reminding, instructing and/or recording (e.g. logging) product application for respective treatment sessions; —subsequent (e.g. one or more follow up) skin diagnostics; and present results (e.g. comparative results); such as in accordance with a treatment plan schedule to monitor progress of a skin treatment plan. Any of these activities may generate data which may be stored remotely for example for user 610 to review, for another individual to review, for aggregation with other user's data to measure treatment plan efficacy, etc. In an embodiment, there is thus provided a system comprising: circuitry using one or more cycleGANs (Cycle-Consistent Generative Adversarial Networks) for translating a RGB (red, green blue) domain skin image to UV (ultraviolet) domain skin image; circuitry using one or more convolutional neural network image classifiers for determining a pixel-wise prediction score for a presence, absence, or severity of one or more skin damage characteristics in the translated UV domain skin image; circuitry for determining at least one skincare product ingredient based on the predicted score for a presence, absence, or severity of one or more skin damage characteristics in the translated UV skin image; and circuitry for transmitting the determined at least one skincare product ingredient to a skincare product formulation device for creation of a custom skincare product.

In an embodiment, there is thus provided a system comprising: a RGB (red, green blue) skin image to UV (ultraviolet) skin image transform engine including computational circuitry configured to perform an RGB skin image to UV skin image translation via one or more cycleGANs (Cycle-Consistent Generative Adversarial Networks); a skin damage prediction engine including computational circuitry configured to determine a pixel-wise prediction score for a presence, absence, or severity of one or more skin damage characteristics in the translated UV skin image using one or more convolutional neural network image classifiers; and a skin damage severity engine including computational circuitry configured to generate a virtual display including one or more instances of a predicted presence, absence, or severity of the one or more skin damage characteristics responsive to one or more inputs based on the prediction scores for the presence, absence, or severity of at least one skin damage characteristic.

Comparative results (e.g. before and after results) may be presented via computing device 602 whether during and/or at the completion, etc, of a treatment plan. As noted, aspects of skin diagnostics may be performed on computing device 602 or by a remotely coupled device (e.g. a server in the cloud or another arrangement).

FIG. 7 is a block diagram of computing device 602, in accordance with one or more aspects of the present disclosure. Computing device 602 comprises one or more processors 702, one or more input devices 704, a gesture-based I/O device 706, one or more communication units 708 and one or more output devices 710. Computing device 602 also includes one or more storage devices 712 storing one or more modules and/or data. Modules may include deep neural net-work model 714, application 716 having components for a graphical user interface (GUI 718) and/or workflow for treatment monitoring (e.g. treatment monitor 720), image acquisition 722 (e.g. an interface) and treatment/product selector 730 (e.g. an interface). Data may include one or more images for processing (e.g. image 724), skin diagnosis data (e.g. respective scores, ethnicity or other user data), treatment data 728 such as logging data related to specific treatments, treatment plans with schedules such as for reminders, etc.)

Application 716 provides the functionality to acquire one or more images such as a video and process the images. In an embodiment, the image is processed using one of models 714 such as generator A (102A) to determine a UV image. In an embodiment, the UV image is processed for skin diagnosis using a deep neural network as provided by another of neural network models 714. Network model may be configured as a model to perform skin diagnosis from an image such as an UV image. In another example, the network model is remotely located (e.g. in server or other computing device). Computing device 602, via application 716, may communicate the UV image for processing and return of skin diagnosis data. Application 716 may be configured to perform the previously described activities.

Storage device(s) 712 may store additional modules such as an operating system 732 and other modules (not shown) including communication modules; graphics processing modules (e.g. for a GPU of processors 702); map module; contacts module; calendar module; photos/gallery module; photo (image/media) editor; media player and/or streaming module; social media applications; browser module; etc. Storage devices may be referenced as storage units herein.

Communication channels 738 may couple each of the components 702, 704, 706, 708, 710, 712, and any modules 714, 716 and 732 for inter-component communications, whether communicatively, physically and/or operatively. In some examples, communication channels 738 may include a system bus, a network connection, an inter-process communication data structure, or any other method for communicating data.

The one or more processors 702 may implement functionality and/or execute instructions within computing device 602. For example, processors 702 may be configured to receive instructions and/or data from storage devices 712 to execute the functionality of the modules shown in FIG. 7, among others (e.g. operating system, applications, etc.) Computing device 602 may store data/information to storage devices 712. Some of the functionality is described further herein be-low. It is understood that operations may not fall exactly within the modules 714, 716 and 732 of FIG. 7 such that one module may assist with the functionality of another.

Computer program code for carrying out operations may be written in any combination of one or more programming languages, e.g., an object oriented programming language such as Java. Smalltalk, C++ or the like, or a conventional procedural programming language, such as the “C” programming language or similar programming languages.

Computing device 602 may generate output for display on a screen of gesture-based I/O device 706 or in some examples, for display by a projector, monitor or other display device. It will be understood that gesture-based I/O device 606 may be configured using a variety of technologies (e.g. in relation to input capabilities: resistive touchscreen, a surface acoustic wave touchscreen, a capacitive touchscreen, a projective capacitance touchscreen, a pressure-sensitive screen, an acoustic pulse recognition touchscreen, or another presence-sensitive screen technology; and in relation to output capabilities: a liquid crystal display (LCD), light emitting diode (LED) display, organic light-emitting diode (OLED) display, dot matrix display, e-ink, or similar monochrome or color display).

In the examples described herein, gesture-based I/O device 706 includes a touchscreen device capable of receiving as input tactile interaction or gestures from a user interacting with the touchscreen. Such gestures may include tap gestures, dragging or swiping gestures, flicking gestures, pausing gestures (e.g. where a user touches a same location of the screen for at least a threshold period of time) where the user touches or points to one or more locations of gesture-based I/O device 706. Gesture-based I/O device 706 and may also include non-tap gestures. Gesture-based I/O device 706 may output or display information, such as graphical user inter-face, to a user. The gesture-based I/O device 706 may present various applications, functions and capabilities of the computing device 602 including, for example, application 716 to acquire images, view images, process the images and display new images, messaging applications, telephone communications, contact and calendar applications, Web browsing applications, game applications, e-book applications and financial, payment and other applications or functions among others.

Although the present disclosure illustrates and discusses a gesture-based V/O device 706 primarily in the form of a display screen device with I/O capabilities (e.g. touchscreen), other examples of gesture-based I/O devices may be utilized which may detect movement and which may not comprise a screen per se. In such a case, computing device 602 includes a display screen or is coupled to a display apparatus to present new images and GUIs of application 716. Computing device 602 may receive gesture-based input from a track pad/touch pad, one or more cameras, or another presence or gesture sensitive input device, where presence means presence aspects of a user including for example motion of all or part of the user.

One or more communication units 708 may communicate with external devices (e.g. server 608, server 410, second computing device 612) such as for the purposes as described and/or for other purposes (e.g. printing) such as via communications network 606 by transmitting and/or receiving network signals on the one or more networks. The communication units may include various antennae and/or network interface cards, chips (e.g. Global Positioning Satellite (GPS)), etc. for wireless and/or wired communications.

Input devices 704 and output devices 710 may include any of one or more buttons, switches, pointing devices, cameras, a keyboard, a microphone, one or more sensors (e.g. biometric, etc.), a speaker, a bell, one or more lights, a haptic (vibrating) device, etc. One or more of same may be coupled via a universal serial bus (USB) or other communication channel (e.g. 738). A camera (an input device 804) may be front-oriented (i.e. on a same side as) to permit a user to capture image(s) using the camera while looking at the gesture based I/O device 706 to take a “selfie”.

The one or more storage devices 712 may take different forms and/or configurations, for example, as short-term memory or long-term memory. Storage devices 712 may be configured for short-term storage of information as volatile memory, which does not retain stored contents when power is removed. Volatile memory examples include random access memory (RAM), dynamic random access memory (DRAM), static random access memory (SRAM), etc. Storage devices 712, in some examples, also include one or more computer-readable storage media, for example, to store larger amounts of information than volatile memory and/or to store such information for long term, retaining information when power is removed. Non-volatile memory examples include magnetic hard discs, optical discs, floppy discs, flash memories, or forms of electrically programmable memory (EPROM) or electrically erasable and programmable (EEPROM) memory.

Though not shown, a computing device may be configured as a training environment to train neural network model 714 for example using the network as shown in FIG. 1A along with appropriate training and/or testing data.

The deep neural network may be adapted to a light architecture for a computing device that is a mobile device (e.g. a smartphone or tablet) having fewer processing resources than a “larger” device such as a laptop, desktop, workstation, server or other comparable generation computing device.

It is understood that second computing device 612 may be similarly configured as computing device 602. Second computing device 612 may have GUIs such as to request and display image(s) and skin sign diagnoses from data stored at server 608 for different users, etc.

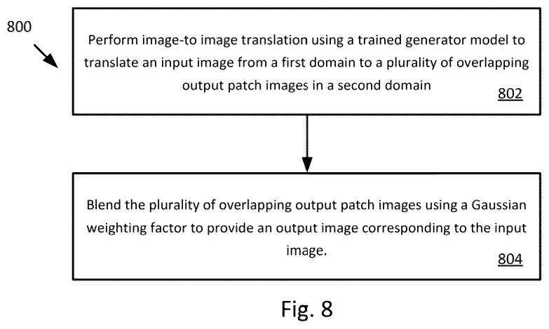

FIG. 8 is a flowchart of operations 800 such as for performance by a computing device in accordance with an embodiment. In an example, the computing device is any one of the devices 602, 612, or 608/610. At 802 operations perform image-to image translation using a trained generator model to translate an input image from a first domain to a plurality of overlapping output patch images in a second domain. And at 804, operations blend the plurality of overlapping output patch images using a Gaussian weighting factor to provide an output image corresponding to the input image.

### This Disclosure Encompasses the Following Embodiments

Embodiment 1: A method comprising: performing image-to image translation using a trained generator model to translate an input image from a first domain to a plurality of overlapping output patch images in a second domain; and blending the plurality of overlapping output patch images using a Gaussian weighting factor to provide an output image corresponding to the input image.

Embodiment 2: The method of embodiment 1, wherein respective pixels of the plurality of overlapping output patch images having a same corresponding pixel location in the input image are all blended using the Gaussian weighting factor to merge the respective pixels to create a single pixel in the output image.

Embodiment 3: The method of embodiment 1 or 2, wherein the Gaussian weighting factor is a weighted vector that weighs the importance of a patch pixel in an overlapping output patch image based on a respective distance between a location of the patch pixel to a center of the overlapping output patch image.

Embodiment 4: The method of embodiment 3 comprising determining the weighted vector from a Gaussian mask having a patch size corresponding to a patch size of an overlapping output patch image, the Gaussian mask generated with a mean about its center and using a variance a.

Embodiment 5: The method of any one of embodiment 1 to 4, wherein the trained generator is applied to a plurality of overlapping input patch images extracted from the input image to produce the plurality of overlapping output patch images.

Embodiment 6: The method of any one of embodiment 1 to 5, wherein the first domain comprises RGB images defined according to a RGB (red, green blue) color model and the second domain comprises ultraviolet images defined according to a grayscale model and wherein the trained generator is a GANs-based generator trained to synthesize UV output images from RGB input images.

Embodiment 7: The method of embodiment 6, wherein the input image is an image of skin and the method comprises using the output image for a diagnostics operation to obtain a skin analysis result for a skin condition or an injury.

Embodiment 8: The method of embodiment 7, comprising any one or more of: providing a treatment product selector responsive to the skin analysis result to obtain a recommendation for at least one of a product and a treatment plan; providing an e-commerce interface to purchase products associated with skin conditions or injuries; providing an image acquisition function to receive the input image; providing a treatment monitor to monitor treatment for at least one skin condition or injury; providing an interface to remind, instruct and/or record treatment activities associated with a product application for respective treatment sessions; and processing a second input image using the trained generator and blending using the Gaussian weighting factor to generate a subsequent UV output image, the second input image capturing a skin condition subsequent to a treatment session; obtaining a subsequent skin analysis result from the subsequent UV output image; and providing a presentation of comparative results using the subsequent skin diagnoses.

Embodiment 9: A method comprising: blending overlapping patch images generated by a GANs-based generator (the blending performed) to reduce one or more of gridding and color inaccuracy effects, wherein respective pixels, from the plurality of overlapping patch images, having a same corresponding pixel location in the input image are all blended using a Gaussian weighting factor to merge the respective pixels to create a single pixel in the output image. Embodiments 1-8 apply to embodiment 9 with adaptation as may be applicable. For example, regarding the application of embodiment 1, the GANs-based generator is trained to translate images from a first domain to images in a second domain; and wherein the method of embodiment 9 comprises extracting from an input image in the first domain a plurality of overlapping input patch images to apply to the GANs-based generator.

Embodiment 10: A system comprising: a RGB (red, green blue) skin image to UV (ultraviolet) skin image transform engine including computational circuitry configured to perform an RGB skin image to UV skin image translation via one or more cycleGANs (Cycle-Consistent Generative Adversarial Networks); a skin damage prediction engine including computational circuitry configured to determine a pixel-wise prediction score for a presence, absence, or severity of one or more skin damage characteristics in the translated UV skin image using one or more convolutional neural network image classifiers; and a skin damage severity engine including computational circuitry configured to generate a virtual display including one or more instances of a predicted presence, absence, or severity of the one or more skin damage characteristics responsive to one or more inputs based on the prediction scores for the presence, absence, or severity of at least one skin damage characteristic.

Embodiment 11: A system comprising: circuitry using one or more cycleGANs (Cycle-Consistent Generative Adversarial Networks) for translating a RGB (red, green blue) domain skin image to UV (ultraviolet) domain skin image; circuitry using one or more convolutional neural network image classifiers for determining a pixel-wise prediction score for a presence, absence, or severity of one or more skin damage characteristics in the translated UV domain skin image; circuitry for determining at least one skincare product ingredient based on the predicted score for a presence, absence, or severity of one or more skin damage characteristics in the translated UV skin image; and circuitry for transmitting the determined at least one skincare product ingredient to a skincare product formulation device for creation of a custom skincare product.

Other embodiments such as computer method, computing device, and computing program product embodiments, for example, corresponding to any of the Embodiments 1-11, etc. are also disclosed.

Practical implementation may include any or all of the features described herein. These and other aspects, features and various combinations may be expressed as methods, apparatus, systems, means for performing functions, program products, and in other ways, combining the features described herein. A number of embodiments have been described. Nevertheless, it will be understood that various modifications can be made without departing from the spirit and scope of the processes and techniques described herein. In addition, other steps can be provided, or steps can be eliminated, from the described process, and other components can be added to, or removed from, the described systems. Accordingly, other embodiments are within the scope of the following claims.

While the computing devices are describe with reference to processors and instructions that when execute cause the computing devices to perform operations, it is understood that other types of circuitry than programmable processors can be configured. Hardware components comprising specifically designed circuits can be employed such as but not limited to an application specific integrated circuit (ASIC) or other hardware designed to perform specific functions, which may be more efficient in comparison to a general purpose central processing unit (CPU) programmed using software. Thus, broadly herein an apparatus aspect relates to a system or device having circuitry (sometimes references as computational circuitry) that is configured to perform certain operations described herein, such as but not limited to those of a method aspect herein, whether the circuitry is configured via programming or via its hardware design.

Throughout the description and claims of this specification, the word “comprise” and “contain” and variations of them mean “including but not limited to” and they are not intended to (and do not) exclude other components, integers or steps. Throughout this specification, the singular encompasses the plural unless the context requires otherwise. In particular, where the indefinite article is used, the specification is to be understood as contemplating plurality as well as singularity, unless the context requires otherwise.

Features, integers characteristics, compounds, chemical moieties or groups described in conjunction with a particular aspect, embodiment or example of the invention are to be understood to be applicable to any other aspect, embodiment or example unless incompatible therewith. All of the features disclosed herein (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings) or to any novel one, or any novel combination, of the steps of any method or process disclosed.

What is claimed is:

1. A computer-implemented method comprising executing on a processor the steps of:
performing image-to image translation using a trained generator model to translate an input image from a first domain to a plurality of overlapping output patch images in a second domain; and
blending the plurality of overlapping output patch images using a Gaussian weighting factor to provide an output image corresponding to the input image.

2. The method of claim 1, wherein respective pixels of the plurality of overlapping output patch images having a same corresponding pixel location in the input image are all blended using the Gaussian weighting factor to merge the respective pixels to create a single pixel in the output image.

3. The method of claim 2, wherein the Gaussian weighting factor is a weighted vector that weighs the importance of a patch pixel in an overlapping output patch image based on a respective distance between a location of the patch pixel to a center of the overlapping output patch image.

4. The method of claim 3 comprising determining the weighted vector from a Gaussian mask having a patch size corresponding to a patch size of an overlapping output patch image, the Gaussian mask generated with a mean about its center and using a variance σ.

5. The method of claim 1, wherein the trained generator is applied to a plurality of overlapping input patch images extracted from the input image to produce the plurality of overlapping output patch images.

6. The method of claim 1, wherein the first domain comprises RGB images defined according to a RGB (red, green blue) color model and the second domain comprises ultraviolet images defined according to a grayscale model and wherein the trained generator is a GANs-based generator trained to synthesize UV output images from RGB input images.

7. The method of claim 6, wherein the input image is an image of skin and the method comprises using the output image for a diagnostics operation to obtain a skin analysis result for a skin condition or an injury.

8. The method of claim 7 comprising any one or more of:
providing a treatment product selector responsive to the skin analysis result to obtain a recommendation for at least one of a product and a treatment plan; and
providing an e-commerce interface to purchase products associated with skin conditions or injuries.

9. The method of claim 7 comprising any one or more of:
providing an image acquisition function to receive the input image;
providing a treatment monitor to monitor treatment for at least one skin condition or injury.
providing an interface to remind, instruct and/or record treatment activities associated with a product application for respective treatment sessions;
processing a second input image using the trained generator and blending using the Gaussian weighting factor to generate a subsequent output image, the second input image capturing a skin condition subsequent to a treatment session; obtaining a subsequent skin analysis result from the subsequent output image; and providing a presentation of comparative results using the subsequent skin diagnoses.

10. A computing device comprising a processor and a memory storing instructions that when executed by the processor cause the computing device to:
perform image-to image translation using a trained generator model to translate an input image from a first domain to a plurality of overlapping output patch images in a second domain; and
blend the plurality of overlapping output patch images using a Gaussian weighting factor to provide an output image corresponding to the input image.

11. The computing device of claim 10, wherein respective pixels of the plurality of overlapping output patch images having a same corresponding pixel location in the input image are all blended using the Gaussian weighting factor to merge the respective pixels to create a single pixel in the output image.

12. The computing device of claim 11, wherein the Gaussian weighting factor is a weighted vector that weighs the importance of a patch pixel in an overlapping output patch image based on a respective distance between a location of the patch pixel to a center of the overlapping output patch image.

13. The computing device of claim 12, wherein the instructions cause the computing device to determine the weighted vector from a Gaussian mask having a patch size corresponding to a patch size of an overlapping output patch image, the Gaussian mask generated with a mean about its center and using a variance σ.

14. The computing device of claim 10, wherein the instructions cause the computing device to extract a plurality of overlapping input patch images extracted from the input image for application to the trained generator to produce the plurality of overlapping output patch images.

15. The computing device of claim 10, wherein the first domain comprises RGB images defined according to a RGB (red, green blue) color model and the second domain comprises ultraviolet images defined according to a grayscale model and wherein the trained generator is a GANs-based generator trained to synthesize UV output images from RGB input images.

16. The computing device of claim 15, wherein the input image is an image of skin and the the instructions cause the computing device to provide the UV output image for a diagnostics operation to obtain a skin analysis result for a skin condition or an injury.

17. The computing device of claim 16, wherein the instructions cause the computing device to perform any one or more of:
providing a treatment product selector responsive to the skin analysis result to obtain a recommendation for at least one of a product and a treatment plan; and
providing an e-commerce interface to purchase products associated with skin conditions or injuries.

18. A computer-implemented method comprising executing on a processor the steps of:
blending overlapping patch images generated by a GAN-based generator to reduce one or more of gridding and color inaccuracy effects, wherein respective pixels, from the plurality of overlapping patch images, having a same corresponding pixel location in the input image are all blended using a Gaussian weighting factor to merge the respective pixels to create a single pixel in the output image.

19. The method of claim 19 comprising determining the weighted vector from a Gaussian mask having a patch size corresponding to a patch size of an overlapping output patch image, the Gaussian mask generated with a mean about its center and using a variance σ.

20. The method of claim 18, wherein the GANs-based generator is trained to translate images from a first domain to images in a second domain; and wherein the method comprises extracting from an input image in the first domain a plurality of overlapping input patch images to apply to the GANs-based generator.

21. A system comprising:
a RGB (red, green blue) skin image to UV (ultraviolet) skin image transform engine including computational circuitry configured to perform an RGB skin image to UV skin image translation via one or more cycleGANs (Cycle-Consistent Generative Adversarial Networks);
a skin damage prediction engine including computational circuitry configured to determine a pixel-wise prediction score for a presence, absence, or severity of one or more skin damage characteristics in the translated UV skin image using one or more convolutional neural network image classifiers; and
a skin damage severity engine including computational circuitry configured to generate a virtual display including one or more instances of a predicted presence, absence, or severity of the one or more skin damage characteristics responsive to one or more inputs based on the prediction scores for the presence, absence, or severity of at least one skin damage characteristic.

22. A system comprising:
circuitry using one or more cycleGANs (Cycle-Consistent Generative Adversarial Networks) for translating a RGB (red, green blue) domain skin image to UV (ultraviolet) domain skin image;
circuitry using one or more convolutional neural network image classifiers for determining a pixel-wise prediction score for a presence, absence, or severity of one or more skin damage characteristics in the translated UV domain skin image;
circuitry for determining at least one skincare product ingredient based on the predicted score for a presence, absence, or severity of one or more skin damage characteristics in the translated UV skin image; and
circuitry for transmitting the determined at least one skincare product ingredient to a skincare product formulation device for creation of a custom skincare product.
